(19) United States
(12) Patent Application Publication
(10) Pub. No.: US 2008/0058555 A1
(43) Pub. Date: Mar. 6, 2008

(54) PREPARATION OF HIGH ASSAY DECABROMODIPHENYL OXIDE

(75) Inventors: Arthur G. Mack, Prairieville, LA (US); Bonnie Gary McKinnie, Magnolia, AR (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 11/843,735

(22) Filed: Aug. 23, 2007

Related U.S. Application Data
(60) Provisional application No. 60/823,817, filed on Aug. 29, 2006.

(51) Int. Cl. C07C 41/22

(52) U.S. Cl. 568/639

(57) ABSTRACT

A process is described for producing a reaction-derived decabromodiphenyl oxide product of high purity. In a continuous bromination process, partially brominated diphenyl oxide and coproduct hydrogen bromide are formed by bringing together elemental bromine and diphenyl oxide continuously in a first reaction zone. The partially brominated diphenyl oxide formed has an average of about 2-6 bromine atoms per molecule. The vapor phase and the partially brominated diphenyl oxide are removed continuously from the first reaction zone as separate entities. Then, or after storage, partially brominated diphenyl oxide is fed to a second reaction zone. This zone contains a refluxing reaction mixture comprising (i) an excess of bromine and (ii) a catalytic quantity of Lewis acid bromination catalyst. As the reaction in this zone is taking place, hydrogen bromide coproduct is removed therefrom in a sufficient amount to form a reaction-derived decabromodiphenyl oxide product of high purity.

### REFERENCE TO RELATED APPLICATION

This application claims the benefit and priority of U.S. Provisional Application No. 60/823,817, filed Aug. 29, 2006, the disclosure of which is incorporated herein by reference.

### TECHNICAL FIELD

This invention relates to improvements in the preparation of decabromodiphenyl oxide products, and more particularly to process technology for producing decabromodiphenyl oxide of high purity.

### BACKGROUND

Decabromodiphenyl oxide (DBDPO) is a time-proven flame retardant for use in many flammable macromolecular materials, e.g. thermoplastics, thermosets, cellulosic materials and back coating applications.

The prior art describes many different processes for producing DBDPO. Despite these prior efforts, apparently it was not possible to produce high purity DBDPO (e.g., DBDPO of a purity of greater than 90%), especially on an industrial scale, unless recrystallization or like expensive purification procedures are utilized.

DBDPO is presently sold as a powder derived from the bromination of diphenyl oxide (DPO) or a partially brominated DPO containing an average of about 0.7 bromine atom per molecule of DPO. Such bromination is conducted in excess bromine and in the presence of a bromination catalyst, usually AlCl₃. The operation is typically conducted at 177° F. (ca. 80.5° C.) with a 2-3 hour feed time. The powdered products are not 100% DBDPO, but rather are mixtures that contain up to about 98% DBDPO and about 1.5%, or a little more, of nonabromodiphenyl oxide co-product. As a partially brominated product, this amount of nonabromodiphenyl oxide is considered problematic by some environmental entities.

It would therefore be desirable to provide process technology, making it possible to prepare highly pure DBDPO on an industrial scale with improved plant throughput. Besides eliminating the need for use of such purification procedures as recrystallization or chromatographic purification, it would be highly desirable to be able to produce high purity DBDPO with increased plant throughput. Highly desirable products would be products comprising (i) at least 99% of DBDPO and (ii) nonabromodiphenyl oxide in an amount not exceeding 0.5%, preferably not exceeding 0.3%, and still more preferably, not exceeding about 0.1%. It would be especially desirable if one could produce DBDPO products comprising (i) at least 99.5% of DBDPO and (ii) nonabromodiphenyl oxide in an amount not exceeding 0.5%, preferably not exceeding 0.3%, and still more preferably, not exceeding about 0.1% on an industrial scale with improved plant throughput for a plant of any given plant capacity.

### BRIEF SUMMARY OF THE INVENTION

This invention provides improved industrially feasible process technology for producing DBDPO with improved plant throughput. Indeed, the process technology of this invention is deemed to enable formation of DBDPO products of greater than 99% purity comprising (i) at least 99.5% of DBDPO and (ii) nonabromodiphenyl oxide in an amount not exceeding 0.5%, preferably not exceeding 0.3%, and still more preferably, not exceeding about 0.1% on an industrial scale with improved plant throughput for a plant of any given plant capacity. And in all cases, the need for recrystallization, chromatographic purification, or like expensive procedures is eliminated pursuant to this invention.

In accordance with one embodiment of this invention, there is provided a process for producing a decabromodiphenyl oxide product of high purity, which process comprises:
A) (1) substantially continuously forming partially brominated diphenyl oxide and coproduct hydrogen bromide by substantially continuously bringing together elemental bromine and diphenyl oxide in a first reaction zone so that a reaction mixture containing partially brominated diphenyl oxide having an average in the range of about 2 to about 6 bromine atoms per molecule is formed, (2) substantially continuously removing a vapor phase comprising coproduct hydrogen bromide from the first reaction zone, and (3) substantially continuously withdrawing from the first reaction zone a reaction product mixture comprising partially brominated diphenyl oxide having in the range of about 2 to about 6 bromine atoms per molecule; and
B) feeding reaction product mixture formed in A) substantially continuously into a second reaction zone containing a refluxing reaction mixture comprising (i) an excess of bromine and (ii) a catalytic quantity of Lewis acid bromination catalyst, and substantially concurrently removing hydrogen bromide coproduct from the second reaction zone in a sufficient amount to form a reaction-derived decabromodiphenyl oxide product of high purity.
   It will of course be understood and appreciated that the terms “first” and “second” as applied to the above reaction zones is merely to distinguish one from the other. In an actual plant facility, for the practice of this invention, there may be several reaction zones, the first of which, for example, could be a reactor in which diphenyl oxide or bromine is produced. Thus, the terms “first” and “second” do not constitute a limitation as regards the position of a reaction zone in a sequential arrangement of operations in a plant facility.

For the purposes of this invention, unless otherwise indicated, the % values given for DBDPO and nonabromodiphenyl oxide are to be understood as being the area % values that are derived from gas chromatography analysis. A procedure for conducting such analyses is presented hereinafter.

Another embodiment of this invention is a process of preparing reaction-derived decabromodiphenyl oxide of high purity, which process comprises:
A) (1) substantially continuously forming partially brominated diphenyl oxide and coproduct hydrogen bromide by substantially continuously bringing together elemental bromine and diphenyl oxide in a first reaction zone so that a reaction mixture containing partially brominated diphenyl oxide having an average in the range of about 2 to about 6 bromine atoms per molecule is formed, (2) substantially continuously removing a vapor phase comprising coproduct hydrogen bromide from the first reaction zone, and (3) substantially continuously withdrawing from the first reaction zone a reaction product mixture comprising partially brominated diphenyl oxide having in the range of about 2 to about 6 bromine atoms per molecule; and
B) maintaining an inversely related time-temperature feed of reaction product mixture formed in A) substantially continuously into a second reaction zone containing a refluxing reaction mixture comprising an excess of bromine containing Lewis acid bromination catalyst, and substantially concurrently reducing the concentration of hydrogen bromide coproduct dissolved in the reaction mixture so that a reaction-derived decabromodiphenyl oxide product of high purity is formed.
   In this embodiment, the higher the temperature of the refluxing reaction mixture, the shorter is the time of the feed (in a batch process) or the shorter is the average residence time of the reaction mixture in the second reaction zone (in a continuous process). Likewise, the lower the temperature of the refluxing reaction mixture, the longer is the time of the feed (in a batch process) or the longer is the average residence time of the reaction mixture in the second reaction zone (in a continuous process).

As used herein including the claims:

1) The term “reaction-derived” means that the composition of the product is reaction determined and not the result of use of downstream purification techniques, such as recrystallization or chromatography, or like procedures that can affect the chemical composition of the product. Adding water or an aqueous base such as sodium hydroxide to the reaction mixture to inactivate the catalyst, and washing away of non-chemically bound impurities by use of aqueous washes such as with water or dilute aqueous bases are not excluded by the term “reaction-derived”. In other words, the products are directly produced in the synthesis process without use of any subsequent procedure to remove or that removes nonabromodiphenyl oxide from decabromodiphenyl oxide.

2) The term “high purity” means that the reaction-derived DBDPO product comprises more than 99% of DBDPO and nonabromodiphenyl oxide in an amount of less than 1% with, if any, a trace of octabromodiphenyl oxide. Preferably the process forms a reaction-derived product which comprises (i) at least 99.5% of DBDPO and (ii) nonabromodiphenyl oxide in an amount not exceeding 0.5%, preferably not exceeding 0.3%, and still more preferably, not exceeding about 0.1%.

3) The terms “substantially continuously” and “substantially continuous” mean that the operation referred to is carried out on a totally continuous basis with no interruptions whatever or that the operation referred to is interrupted one or more times as long as such interruptions are of short enough duration as not to affect in any significant way the end result of producing a reaction-derived DBDPO product of high purity.

4) The term “substantially concurrently reducing” as regards the amount of hydrogen bromide means that the reducing is taking place at exactly the same time or at substantially the same time that the feeding is taking place. Likewise the term “substantially concurrently feeding” means that the feeding is taking place at exactly the same time or at substantially the same time that the reducing of the amount of hydrogen bromide is taking place. It is to be clearly understood that the feeding and the reducing of the amount of hydrogen bromide need not start at the same moment in time. For example, there can be a time lag between the commencement of the feed and the evolution of enough hydrogen bromide to initiate the reducing of the amount thereof in the reactor. Likewise, if and when the feeding is terminated, there can be a period of time thereafter during which the amount of hydrogen bromide in the reactor can be reduced. In addition, it should be understood that the terms “substantially concurrently reducing” and “substantially concurrently feeding” include one or more interruptions in such operations as long as such interruptions are of short enough duration as not to affect in any significant way the end result of producing a reaction-derived DBDPO product of high purity.

The reaction product mixture formed in step A) can be stored until needed as feed in step B), or such reaction product mixture can be directly fed in to the second reaction zone pursuant to step B). Alternatively, a portion of the reaction product mixture formed in step A) can be directly fed into the second reaction zone pursuant to step B) with the remainder of such reaction product mixture being stored for subsequent use as feed to step B). The reaction product mixture formed in step A) can be a solids-free liquid phase reaction product mixture or it can be in the form of a solids-containing liquid phase reaction product mixture, such as a slurry.

In order to produce high purity reaction-derived DBDPO product, the mixture fed in B) consists essentially of (i) elemental bromine and (ii) partially brominated diphenyl oxide. This means that such liquid phase mixture is either completely devoid of hydrogen bromide or contains an amount of hydrogen bromide that is sufficiently small as not to preclude the formation in step B) of a reaction-derived DBDPO product of high purity.

The reaction in B) can be conducted as a batch process or as a continuous process. In either case, the production rate in any given plant can be increased over conventional prior art processes. Thus, in accordance with another of the embodiments of this invention, there is provided an improved batch process. This process comprises a process as described above wherein B) is conducted as a batch process wherein the amount of bromine present in the second reaction zone is at least sufficient to maintain a stoichiometric excess in the second reaction zone, preferably without replenishment during the process. However, additional bromine can be fed into the second reaction zone before and/or during the feed of reaction product mixture formed in A). Pursuant to another of the embodiments of this invention, there is provided an overall continuous process. In this overall continuous process, B) above is conducted as a continuous operation such that:
reaction product mixture formed in A) is substantially continuously fed into the second reaction zone;
reaction mixture comprising decabromodiphenyl oxide product, bromine, and catalyst substantially continuously exits from the second reaction zone;
a separate vapor phase composed of hydrogen bromide and bromine substantially continuously exits from the second reaction zone, this vapor phase is passed through a condensing system so that bromine is liquefied and returned to the second reaction zone and hydrogen bromide passes through the condensing system and is subsequently recovered, e.g., in a scrubber system containing, say, water whereby hydrobromic acid is formed, or aqueous base such as a NaOH or KOH whereby a metal bromide salt is formed.
Lewis acid bromination catalyst and additional bromine are fed (i) periodically or continuously, and (ii) individually or in admixture into the second reaction zone separately and apart from the feed of reaction product mixture formed in A), and
the amount of additional Lewis acid bromination catalyst fed into the second reaction zone is an amount that substantially continuously maintains a catalytic amount of Lewis acid bromination catalyst in the second reaction zone and the amount of bromine fed into the second reaction zone is an amount that substantially continuously maintains an excess of bromine in the second reaction zone. Desirably, this excess is an excess in the range of about 50 to about 150 mole percent more than the amount theoretically required to perbrominate the partially brominated diphenyl oxide.

The above and other embodiments and features of this invention will become still further apparent from the ensuing description and appended claims.

### BRIEF DESCRIPTION OF THE DRAWINGS

FIG. 1 is a copy of the GC trace of the product formed in Example 1 hereinafter.

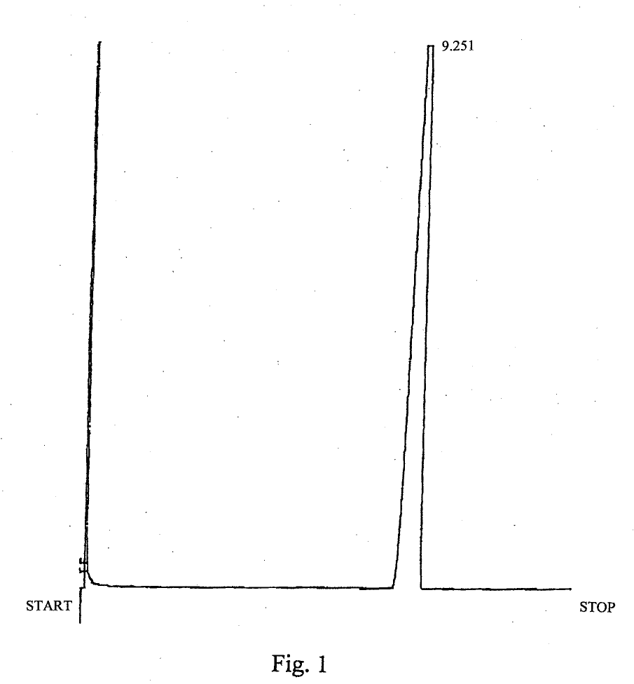

FIG. 2 is a copy of the GC trace of the product formed in Example 2 hereinafter.

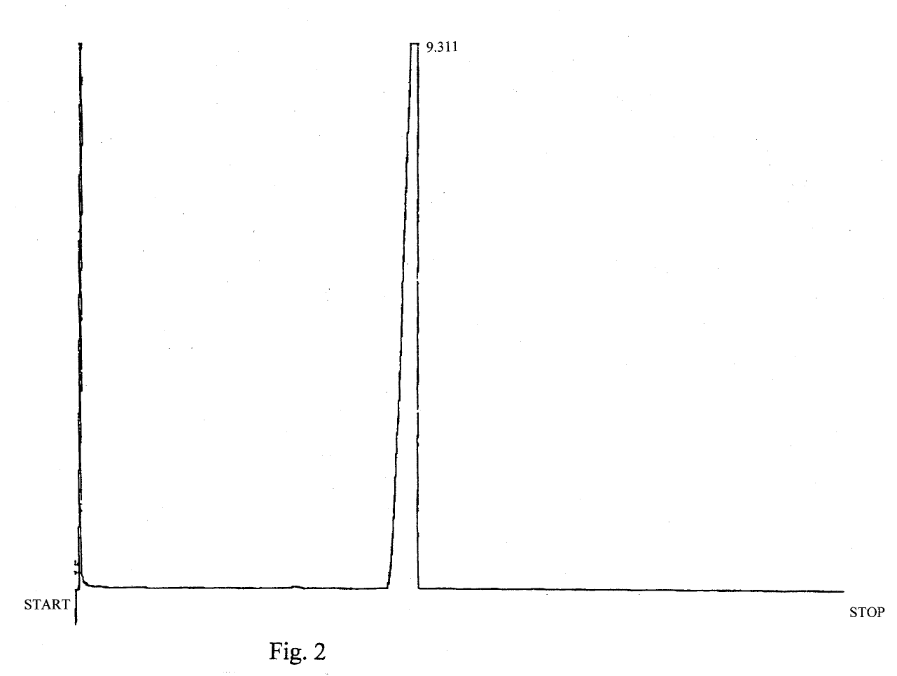

### FURTHER DETAILED DESCRIPTION OF EMBODIMENTS OF THE INVENTION

As noted above the process technology of this invention comprises at least steps A) and B).

### Step A)

Step A) is conducted as a continuous process. Thus the rate of feed to the first reaction zone and the rate of removal of the reaction product from the first reaction zone should be maintained such that the quantity of reaction mixture within the first reaction zone remains substantially constant.

The reaction in A) can be conducted in the presence or absence of a catalyst. If a catalyst is used in A) it is important that the reaction proceeds to form a partially brominated diphenyl oxide product having an average in the range of about 2 to about 6, preferably in the range of about 3 to about 5, and more preferably about 4 bromine atoms per molecule. When conducting the reaction of step A) in the absence of a catalyst and at reflux temperatures in the range of about 57 to about 60° C., partially brominated DBDPO having an average of about 4 bromine is typically formed. Preferably, the partially brominated DPO reaction product mixture formed in step A) is a solids-free solution. However, if partially brominated DPO reaction product mixtures formed in step A) in which the average number of bromine atoms per molecule is no more than about 6 bromine atoms per molecule do undergo precipitation formation, such reaction product mixture nevertheless can be used as feed in step B), e.g., as a slurried feed.

It will be understood that the “partially brominated DPO” reaction product formed in step A) can contain some unbrominated DPO and/or some brominated DPO having one bromine atom in the molecule. It will also be understood that the “partially brominated DPO” can be composed entirely or substantially entirely of reaction product having the same number of bromine atoms per molecule in the range of about 2 to about 6, preferably in the range of about 3 to about 5, and more preferably about 4 bromine atoms per molecule.

To achieve the formation of partially brominated DPO, catalyst strength (if used), catalyst concentration (if catalyst is used), reaction temperature, the pressure under which the reaction is conducted, and the average residence time in the reaction zone may all have an effect.

As noted above, step A) can be conducted in the absence of any added catalyst, or a suitable weak Lewis acid catalyst can be used. Any of a variety of such catalysts can be used to prepare partially brominated DPO in step A). The catalysts used can be Lewis acids weaker than aluminum chloride, aluminum bromide, ferric chloride, ferric bromide, gallium chloride, and gallium bromide. When using a catalyst, it is desirable to use a known weaker Lewis acid such as antimony chloride, antimony bromide, zinc chloride, zinc bromide, zirconium tetrachloride, zirconium tetrabromide, titanium tetrachloride, titanium tetrabromide, or other known weaker Lewis acid.

In the absence of a catalyst, the reaction temperature used in A) is generally in the range of about 20 to about 60° C. When a catalyst is used the temperature should be somewhat less than used in the absence of a catalyst, e.g., in the range of about 10 to about 50° C.

Increased reaction pressure tends to increase the extent of bromination. Nevertheless, pursuant to this invention, it is possible to operate at superatmospheric pressures in the range of about 5 to about 40 psig (ca. 1.36×10⁵ to 3.77×10⁵ Pa) provided that the formation of precipitating product solids having more than about 6 bromine atoms per molecule are not formed in the reaction mixture, or at least the amount thereof is kept to a minimum. However, in carrying out step A) the pressure in the first reaction zone is preferably no more than autogenous pressure in a closed reaction system, and more preferably, the pressure in the first reaction zone is at substantially atmospheric pressure. It is also possible to operate at subatmospheric pressures.

The average residence time used in conducting A) above can vary. However, generally speaking, as long as the desired partially brominated DPO is formed, the shorter the residence time, the better. Accordingly, the average residence times in the first reaction zone are typically in the range of about 5 to about 90 minutes and preferably in the range of about 10 to about 60 minutes.

In the reaction of A), the first reaction zone can be in a partially filled reactor having a vapor space or it can be conducted in a reactor filled with liquid phase reaction mixture which is under autogenous pressure. In the former case, a vapor phase comprising hydrogen bromide is also formed in A) and in order to achieve high purity DBDPO, vapor phase comprising hydrogen bromide is substantially continuously separated from the first reaction zone. In the latter case, the vapor phase is retained in the reaction mixture until release from the first reaction zone. If necessary, steps should be taken to ensure that the feed to the second reaction zone is devoid or substantially devoid of hydrogen bromide, i.e., the lower the amount, if any, the better. By “substantially devoid” is meant that the amount of hydrogen bromide present in the feed to the second reaction zone is sufficiently low as not to preclude the formation of reaction-derived DBDPO of high purity.

While various types of reaction equipment can be utilized in carrying out step A), use of at least one continuously stirred reactor, commonly referred to in the art as a CSTR, is preferred on the basis of economy and process efficiency. Operation in a CSTR which is devoid of added Lewis acid bromination catalyst is especially preferred.

The reaction of step A) can be conducted in various ways. Thus, the DPO can be fed to bromine already present in the first reaction zone, or the bromine can be fed to DPO already present in the first reaction zone. Alternatively, the DPO and the bromine can be fed substantially concurrently into the first reaction zone. Combinations of such feed techniques can be used. If a catalyst is used it can be fed in admixture with the bromine or in admixture with the DPO or the catalyst can be fed separately as a concurrent feed. Combinations of such procedures can also be used. In short, any suitable way of bringing the components together in order to form the partially brominated DPO can be used.

### Step B)

Among the principal features of step B) is that partially brominated DPO formed as in step A) is used as the feed to be brominated. Use of such partially brominated DPO feed enables removal of about 20 to about 60% or about 30 to about 50% or about 49% of the total HBr load in a continuous operation in step A) while separately, and at the same time if desired, conducting in step B) a second bromination reaction in the second reaction zone wherein feed formed in the step A) can be brominated in step B) in a shorter reaction period (if a batch process) or with a shorter residence time (if a continuous process) and in either case, with a reduced total HBr load in step B). Another of the principal features of step B) is that the combination of steps A) and B) produces a reaction-derived DBDPO product of high purity while retaining the ability to accomplish this with higher plant throughput.

In the practice of this invention the processing disclosed in commonly owned copending U.S. Application No. 60/823,811, filed on Aug. 29, 2006, and entitled “Preparation of High-Assay Decabromodiphenyl Oxide” is adapted for use herein in order to achieve preparation of reaction-derived DBDPO product of high purity.

On the basis of studies conducted in our laboratories, one of the prime difficulties in producing high purity DBDPO is the existence of an equilibrium between nonabromodiphenyl oxide and decabromodiphenyl oxide. This equilibrium can be depicted as follows:

Br₉-DPO+Br₂ [symbol] Br₁₀-DPO+HBr

As more fully described in the foregoing commonly owned application, prolonged feed of DPO and/or partially brominated DPO to refluxing bromine while substantially concurrently reducing hydrogen bromide content in the reactor enables a shift to the right in this equilibrium so that the amount of nonabromodiphenyl oxide is diminished and more of the desired decabromodiphenyl oxide forms and precipitates with less nonabromodiphenyl oxide being coprecipitated within the decabromodiphenyl oxide particles. It is also believed that if the DPO and/or partially brominated DPO is fed too rapidly, the precipitation of at least one Brg-DPO isomer occurs so rapidly that the above equilibrium is not totally reached.

Accordingly, step B) is carried out in such a way as to maintain a substantially continuous, coordinated time-temperature feed of partially brominated DPO feed formed as in step A) and thus having an average in the range of about 2 to about 6 bromine atoms per molecule (preferably an average in the range of about 3 to about 5 bromine atoms per molecule, and more preferably an average of about 4 bromine atoms per molecule) to a reactor containing a refluxing reaction mixture comprising an excess of bromine containing Lewis acid bromination catalyst, and substantially concurrently reducing the amount of hydrogen bromide coproduct present in the reactor so that a DBDPO product containing more than 99% of DBDPO is formed in the reactor.

A more particular process utilized herein as a preferred step B) process comprises preparing reaction-derived decabromodiphenyl oxide of high purity by feeding partially brominated diphenyl oxide into the second reaction zone containing a refluxing reaction mixture comprising an excess of bromine containing Lewis acid bromination catalyst. Because pursuant to this invention the product of step A) is used as the feed, the time of the feeding is shortened and thus the overall plant throughput is improved. Pursuant to this invention, a feed period in the range of about 2 to about 12 hours is used. While the feeding is taking place, the content of hydrogen bromide present in the reactor is substantially concurrently reduced so that a high purity decabromodiphenyl oxide product is formed. The feed of the partially brominated diphenyl oxide is substantially continuous. However, it may be possible to use a pulsed feed with suitable intervals of time separating the feeding periods. Such intervals of time separating the pulses of feed should be short enough as not to preclude the preparation of reaction-derived decabromodiphenyl oxide of high purity.

Generally speaking, from the viewpoint of productivity and plant throughput, the shorter the feed period or residence time used, the better. But pursuant to this invention the feed period or residence time used should be sufficiently long at the reaction temperature being used to enable formation of a reaction-derived DBDPO product of high purity.

Therefore, depending on the temperature at which the bromination is occurring, the feed of partially brominated DPO product(s) from step A) should occur during a period in the range of about 2 to about 12 hours, and preferably in the range of about 4 to about 10 hours, with such period being long enough to reach the desired equilibrium state. When operating at a plant scale this period of time in part represents a compromise between rate of reactor throughput and desire for as slow a feed as is practicable for achieving the desired product purity. Thus, the duration of the substantially continuous feed should be a period of time that is prolonged yet consistent with achieving an economically acceptable plant throughput. The use of a slow feed is desirable as it provides a longer period of time for a given quantity of DPO or partially brominated DPO to reach the decabromodiphenyl oxide stage before significant precipitation of nonabromodiphenyl oxide encased in decabromodiphenyl oxide particles takes place.

In practicing a process of this invention it is important to minimize the content of hydrogen bromide present in the reactor. Among various ways of achieving such minimization are the following:

A combination of vigorous refluxing of the bromine in the reactor, withdrawal of the hydrogen bromide vapor phase from the reactor, and efficient condensation of bromine vapors being withdrawn with the hydrogen bromide is desirable and is preferably utilized.

Use of a fractionation column to effectively separate as much HBr from the bromine in the column as feasible. In this way the bromine returning to the reactor carries less, if any, HBr back into the reactor. The fractionation column can be a packed column or it can be free of packing, and should be designed to effect an efficient separation of HBr from bromine.

An inert gas purge of the reactor (e.g., with argon, neon, or preferably nitrogen) to carry away HBr is useful.

Use of bromine in the vapor state as a stripping gas. Besides carrying away HBr, the use of bromine vapors is a way of introducing more heat into the reactor and thereby contributing to more vigorous refluxing within the system.

Operation at atmospheric, subatmospheric or superatmospheric pressures to enable a refluxing condition of the reaction mixture at the selected process temperature.

Since the bromination is conducted in excess refluxing bromine, the reactor is of course equipped with a reflux condenser and preferably a reflux fractionation column. This should be designed to return to the reaction as little HBr in the condensed bromine as is technically and economically feasible under the circumstances.

In all cases, the hydrogen bromide leaving the reaction system is preferably recovered for use or sale. Recovery can be achieved by use of a suitable scrubbing system using one or more aqueous liquid scrubbers such as water, or dilute NaOH or KOH solution.

The relationship between bromination reaction temperature and pressure under which the bromination in b) is being operated is worthy of comment. Ideally it is desirable to operate at as high a temperature as possible and as low a pressure as possible to adequately reduce the HBr concentration in the bromine as more HBr is removed from the reactor. Sampling a refluxing bromination reaction mixture of this type in order to assay the percentage of HBr dissolved in the Br₂ at any given time is not deemed feasible when using ordinary laboratory or plant equipment. Such sampling requires special equipment such as built-in stationary probes to periodically remove representative samples of the reaction mixture from the reactor. Thus when using ordinary plant equipment, operation at maximum temperature and minimum pressure is desirable as a way of reducing the HBr concentration in the bromine. However, maintaining a high reaction temperature in such a reaction system is not as easy as it might appear. For one thing, considerable heat input is required to the reaction mixture, and this can impose limitations in existing plant equipment. Consequently, in most cases it is desirable when operating on a commercial scale to conduct the reaction at a mildly elevated pressure (e.g., in the range of about 5 to about 20 psig (ca. 1.36×10⁵ to 2.39×10⁵ Pa)), and having the temperature high enough to effect vigorous refluxing to thereby keep the HBr concentration in the bromine low as more HBr is removed from the reactor.

As noted above the process technology of this invention enables the preparation of highly pure DBDPO products while at the same time achieving improved plant throughput. For example, as seen from the Examples herein, DBDPO products having a purity of 100% as indicated by GC analysis have been prepared pursuant to this invention. Such products can be said to be “reaction-derived” since they are reaction determined and not the result of use of downstream purification techniques, such as recrystallization, chromatography, or like procedures. In other words, the products are of high purity.

The partially brominated DPO can be fed as solids, but preferably the feed is in molten form or as a solution in a solvent such as methylene bromide or bromoform. To prevent freeze up in the feed conduit, the partially brominated DPO is desirably fed at a temperature that is at least about 20 higher than the melting temperature of the particular partially brominated DPO being fed.

Excess bromine is used in the Lewis acid catalyzed bromination reaction. Enough bromine should be present to provide in the range of about 4 to about 12 moles of excess bromine over the amount required to perbrominate the partially brominated DPO.

As noted above, the refluxing temperature of bromine at atmospheric or slightly elevated pressures is in the range of about 57 to about 59° C. but when conducting B) at higher elevated pressures suitably higher temperatures should be used in order to maintain a vigorous refluxing condition.

If desired, a suitable solvent can be included in the reaction mixtures of B). This can be advantageous in that one can have a higher reaction temperature and possibly a lower HBr concentration in the bromine thereby giving higher purity DBDPO. Among such solvents are methylene bromide and bromoform.

Various iron and/or aluminum Lewis acids can be added to the bromine to serve as the bromination catalyst. These include the metals themselves such as iron powder, aluminum foil, or aluminum powder, or mixtures thereof. Preferably use is made of such catalyst materials as, for example, ferric chloride, ferric bromide, aluminum chloride, aluminum bromide, or mixtures of two or more such materials. More preferred are aluminum chloride and aluminum bromide with addition of aluminum chloride being more preferred from an economic standpoint. It is possible that the makeup of the catalyst may change when contained in a liquid phase of refluxing bromine. For example, one or more of the chlorine atoms of the aluminum chloride may possibly be replaced by bromine atoms. Other chemical changes are also possible. The Lewis acid should be employed in an amount sufficient to effect a catalytic effect upon the bromination reaction being conducted. Typically, the amount of Lewis acid used will be in the range of about 0.06 to about 2 wt %, and preferably in the range of about 0.2 to about 0.7 wt % based on the weight of the bromine being used.

After all the partially brominated DPO is added, the reaction mixture can be kept at reflux for a suitable period of time to ensure completion of the perbromination to DPDPO. A period of up to about one hour can be used. Generally speaking, the benefits of such post-reaction refluxing tend to offset by the prolongation of the overall operation, and thus use of such post reflux, though permissible, is not preferred.

Termination of the bromination reaction is typically effected by deactivating the catalyst with water and/or an aqueous base such as a solution of sodium hydroxide or potassium hydroxide.

When conducting step B) on a continuous basis, after a suitable average residence time in the second reaction zone (e.g., in the range of about 0.2 to about 3 hours), the reaction mixture is substantially continuously withdrawn from the second reaction zone. The feed of the partially brominated DPO can be a substantially continuous feed and bromine remaining associated with the partially brominated DPO product from step A) is co-fed therewith. Whether conducting the process on a batch basis or on a continuous basis it is desirable to substantially continuously separate hydrogen bromide coproduct from the second reaction zone. In addition, hydrogen bromide and liquid phase comprising at least bromine and partially brominated diphenyl oxide substantially continuously leave the first reaction zone.

Since hydrogen bromide is formed as a coproduct in both step A) and step B), two scrubber systems can be employed, one receiving the hydrogen bromide effluent from step A) and the other receiving the hydrogen bromide effluent from step B). It is also possible to utilize one sufficiently large scrubbing system to receive both such effluent streams of hydrogen bromide.

Various types of reaction equipment are known for conducting a continuous reaction with continuous takeoff of a vapor phase component from the reaction mixture and concurrently removing a liquid phase reaction product from the reactor. Typically, such reaction equipment involves use of a refrigerated condenser system such as a refrigerated condenser column. Such columns can be packed columns or they can be devoid of any packing. The vapor phase, which contains material that is to be returned in liquid form to the liquid phase for withdrawal from the reactor—in this case bromine—is condensed by use of refrigeration or other suitable means of cooling in the column. The remainder of the vapor phase which is not condensed—in this case hydrogen bromide—passes through the column as an effluent vapor and is recovered by introduction into a suitable scrubbing system. If the scrubber contains water, the hydrogen bromide is converted to hydrobromic acid. If the scrubber contains an aqueous base such as sodium hydroxide or potassium hydroxide, the hydrogen bromide is converted to sodium bromide or potassium bromide.

### Gas Chromatographic Procedure

The gas chromatography is on a Hewlett-Packard 5890, series II, with Hewlett-Packard model 3396 series II integrator, the software of which is that installed with the integrator by the manufacturer. The gas chromatograph column used is an aluminum clad fused silica column, Code 12 AQ5 HT5 (Serial number A132903) obtained from SGE Scientific, with film thickness of 0.15 micron. The program conditions are: initial start temperature 250° C., ramped up to 300° C. at a rate of 5° C./min. The column head pressure is 10 psig (ca. 1.70×10⁵ Pa). The carrier gas is helium. The injection port temperature is 275° C. and the flame ionization temperature is 325° C. Samples are prepared by dissolving ca. 0.1 g in 8-10 mL of dibromomethane. The injection size is 2.0 microliters.

### EXAMPLES

The practice of embodiments of the invention and advantages achievable by practice of the embodiments of the invention are illustrated in the following Examples. These Examples, which serve as indications of the feasibility of conducting at least step A) as a continuous operation followed by a step B) operation, are not intended to impose limitations on the overall scope of the invention. In these Examples, the first step simulates larger scale operation in a first reaction zone and step B) simulates larger scale operation is a second reaction zone.

### Example 1

### Step A: Formation of Partially Brominated Diphenyl Oxide

A 250-mL four-necked flask equipped with a mechanical stirrer, a glycol-cooled reflux condenser maintained at 0° C., an addition funnel, a thermometer with a temperature regulator and an ice-cold caustic scrubber, was charged with 0.2 mol (34.0 g) of diphenyl oxide. The addition funnel was charged with bromine (1.2 mol, 192 g, approximately 62 mL). Diphenyl oxide was heated to about 25° C. and stirred. With stirring under nitrogen, bromine was now added, drop-wise, to the stirred diphenyloxide over a period of 55 minutes. The reaction mixture was now heated and stirred at 45° C. for another 45 minutes. The reaction mixture was now allowed to cool to room temperature. A drying tube containing Drierite was installed on the condenser and the reaction mixture was stored overnight under nitrogen, for use the next day. The total volume of this solution was approximately 67 mL.

### Step B: Bromination of Partially Brominated Diphenyl Oxide Prepared in Step a, Above

A 1-L four-necked round bottom flask was equipped in a manner identical to what was used in step A, above, except that the addition funnel was also equipped with a Teflon dip tube approximately 1/16″ in diameter and of sufficient length to reach well beneath the bromine surface for sub-surface feeding. Also, a vigreux column, approximately seven inches in length and ½ in. in diameter, was installed on the reactor before the condenser to provide additional fractionation of the liquid and vapor phases. The reactor was charged with bromine (3.97 moles, 635.5 g, approx. 2055 mL), followed by 3.4 g of anhydrous aluminum chloride catalyst. The bromine/catalyst mixture was stirred and heated to 60° C. Partially brominated DPO (prepared before as described in step A, above), was now added, subsurface to bromine/catalyst at 60° C., over a period of about three hours and twenty three minutes. The reaction mixture was allowed to reflux at 60° C. for an additional three hours, while using the same dip tube to allow a slow nitrogen sweep through the reaction mixture. After the reflux time was over, the reaction mixture was allowed to cool to room temperature. Water (250 mL) was now added to decompose the catalyst. Excess bromine was now removed by steam distillation until the vapor temperature of 100° C. was reached. The aqueous slurry of the product was allowed to cool to 40° C. Aqueous sodium hydroxide (50 wt. % solution) was now added until a pH of about 9-10 was reached. The product was now filtered using a sintered glass funnel and the cake was washed once with 200 mL of fresh water. The cake was allowed to dry in air overnight. This gave a shiny crystalline solid powder, weighing 185.9 grams. A GC analysis of the sample indicated the product to be 100 area % decabromodiphenyl oxide. Further analytical analysis of product sample using other protocols confirmed decabromodiphenyl oxide purity of no less than about 99.7 area %.

### Example 2

### Step A: Formation of Partially Brominated Diphenyl Oxide

This step was performed in a manner identical to step A of example 1 as described above, except that a 1-L round bottom flask was used. This flask was charged with 170 g (1.0 mol) of diphenyloxide to which a total of 960 g (309.6 mL) of bromine was fed over a period of 1 hour and thirty eight minutes. The reaction temperature was maintained between 25-35° C. during the addition, followed by a reflux at 50-58° C. for thirty minutes. This reaction mixture was stored overnight as described in part A of Example 1, above. Total volume of this mixture was approximately 300 mL.

### Step B: Bromination of Partially Brominated Diphenyl Oxide

This procedure was also performed in a manner identical to step B as described for example 1 above. The equipment design was also identical to the one used in step B, above. A brief description is as follows:

A 3-L round bottom flask was equipped with a mechanical stirrer, a 7 in.×½ in. vigreux column to which was attached a glycol-cooled reflux condenser, an addition funnel with a 1/16 in. Teflon dip tube for sub-surface feed, a thermometer with a temperature regulator and an ice cold caustic scrubber. The reactor was charged with bromine (19.85 mol, 3177.5 g, 1025 mL) and 17.0 g of anhydrous aluminum chloride catalyst. The bromine/catalyst mix was stirred under nitrogen and heated to 55° C. The addition funnel was charged with partially brominated DPO feed, prepared earlier as described in step A above. Partially brominated DPO was then added, sub-surface, to the reactor containing bromine and catalyst, over a period of 4.5 hours, at a temperature of 55-60° C. The contents were then heated at reflux for an additional two hours. The reaction mixture was now cooled to room temperature and 250 mL of water was added to decompose the catalyst. An exotherm to 43° C. was observed upon water addition. After this another 950 mL of water (total=1200 mL) was added. The reaction mixture was now heated and excess bromine was removed by steam distillation until the vapor temperature of 100° C. was reached. Heat was cut off and the contents were cooled to 30° C. Aqueous caustic (50% aq. NaOH, 45.4 g) was added and stirred well. Filtered the product and the cake was washed with water (3×800 mL), followed by drying in air overnight. This gave a light orange crystalline powder, weighing 952.3 g. A GC analysis of a small sample showed the product to be 100 area % deca. This product was heated in an oven at 210° C. for six hours to give 942.2 g of the finished product. Further analytical analysis of product sample using other protocols confirmed decabromodiphenyl oxide purity of no less than about 99.7 area %.

### DBDPO Products and Flame Retardant Usage

The DBDPO products formed in processes of this invention are white or slightly off-white in color. White color is advantageous as it simplifies the end-user's task of insuring consistency of color in the articles that are flame retarded with the DBDPO products.

The DBDPO products formed in the processes of this invention may be used as flame retardants in formulations with virtually any flammable material. The material may be macromolecular, for example, a cellulosic material or a polymer. Illustrative polymers are: olefin polymers, cross-linked and otherwise, for example homopolymers of ethylene, propylene, and butylene; copolymers of two or more of such alkene monomers and copolymers of one or more of such alkene monomers and other copolymerizable monomers, for example, ethylene/propylene copolymers, ethylene/ethyl acrylate copolymers and ethylene/propylene copolymers, ethylene/acrylate copolymers and ethylene/vinyl acetate copolymers; polymers of olefinically unsaturated monomers, for example, polystyrene, e.g. high impact polystyrene, and styrene copolymers, polyurethanes; polyamides; polyimides; polycarbonates; polyethers; acrylic resins; polyesters, especially poly(ethyleneterephthalate) and poly(butyleneterephthalate); polyvinyl chloride; thermosets, for example, epoxy resins; elastomers, for example, butadiene/styrene copolymers and butadiene/acrylonitrile copolymers; terpolymers of acrylonitrile, butadiene and styrene; natural rubber; butyl rubber and polysiloxanes. The polymer may be, where appropriate, cross-linked by chemical means or by irradiation. The DBDPO products of this invention can be used in textile applications, such as in latex-based back coatings.

The amount of a DBDPO product of this invention used in a formulation will be that quantity needed to obtain the flame retardancy sought. It will be apparent to those skilled in the art that for all cases no single precise value for the proportion of the product in the formulation can be given, since this proportion will vary with the particular flammable material, the presence of other additives and the degree of flame retardancy sought in any given application. Further, the proportion necessary to achieve a given flame retardancy in a particular formulation will depend upon the shape of the article into which the formulation is to be made, for example, electrical insulation, tubing, electronic cabinets and film will each behave differently. In general, however, the formulation, and resultant product, may contain from about 1 to about 30 wt %, preferably from about 5 to about 25 wt % DBDPO product of this invention. Master batches of polymer containing DBDPO, which are blended with additional amounts of substrate polymer, typically contain even higher concentrations of DBDPO, e.g., up to 50 wt % or more.

It is advantageous to use the DBDPO products of this invention in combination with antimony-based synergists, e.g., Sb₂O₃. Such use is conventionally practiced in all DBDPO applications. Generally, the DBDPO products of this invention will be used with the antimony based synergists in a weight ratio ranging from about 1:1 to 7:1, and preferably of from about 2:1 to about 4:1.

Any of several conventional additives used in thermoplastic formulations may be used, in their respective conventional amounts, with the DBDPO products of this invention, e.g., plasticizers, antioxidants, fillers, pigments, UV stabilizers, etc.

Thermoplastic articles formed from formulations containing a thermoplastic polymer and DBDPO product of this invention can be produced conventionally, e.g., by injection molding, extrusion molding, compression molding, and the like. Blow molding may also be appropriate in certain cases.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

Except as may be expressly otherwise indicated, the article “a” or “an” if and as used herein is not intended to limit, and should not be construed as limiting, a claim to a single element to which the article refers. Rather, the article “a” or “an” if and as used herein is intended to cover one or more such elements, unless the text expressly indicates otherwise.

Each and every patent or publication referred to in any portion of this specification is incorporated in toto into this disclosure by reference, as if fully set forth herein.

That which is claimed:

1. A process for producing a reaction-derived decabromodiphenyl oxide product of high purity, which process comprises:
A) (1) substantially continuously forming partially brominated diphenyl oxide and coproduct hydrogen bromide by substantially continuously bringing together elemental bromine and diphenyl oxide in a first reaction zone so that a reaction mixture containing partially brominated diphenyl oxide having an average in the range of about 2 to about 6 bromine atoms per molecule is formed, (2) substantially continuously removing a vapor phase comprising coproduct hydrogen bromide from the first reaction zone, and (3) substantially continuously withdrawing from the first reaction zone a reaction product mixture comprising partially brominated diphenyl oxide having in the range of about 2 to about 6 bromine atoms per molecule; and
B) feeding reaction product mixture formed in A) substantially continuously into a second reaction zone containing a refluxing reaction mixture comprising (i) an excess of bromine and (ii) a catalytic quantity of Lewis acid bromination catalyst, and substantially concurrently reducing the content of hydrogen bromide present in the reactor so that a reaction-derived decabromodiphenyl oxide product of high purity is formed.

2. A process as in claim 1 wherein said reaction product mixture of (3) consists essentially of (i) elemental bromine and (ii) said partially brominated diphenyl oxide, and optionally, (iii) weak Lewis acid bromination catalyst residue.

3. A process as in claim 1 wherein the reaction in A) is conducted in the absence of any added catalyst.

4. A process as in claim 1 wherein the reaction in A) is conducted in the presence of a weak Lewis acid bromination catalyst.

5. A process as in claim 1 wherein said excess of bromine is an excess of bromine in the range of about 50 to about 150 mole percent of the amount required to perbrominate the partially brominated diphenyl oxide.

6. A process as in claim 1 wherein B) is conducted as a batch process wherein the amount of bromine initially present in said second reaction zone is at least sufficient to maintain, without replenishment during the process, an excess of bromine in the range of about 50 to about 150 mole percent of the amount required to perbrominate the partially brominated diphenyl oxide to be fed into said second reaction zone.

7. A process as in claim 1 wherein B) is conducted as a continuous operation such that in B):
reaction product mixture formed in A) is substantially continuously fed into the second reaction zone;
reaction mixture comprising decabromodiphenyl oxide product, bromine, and catalyst substantially continuously exits from the second reaction zone;
a separate vapor phase composed of hydrogen bromide and bromine substantially continuously exits from the second reaction zone, and this vapor phase is passed through a condensing system so that bromine is liquefied and returned to the second reaction zone;
Lewis acid bromination catalyst and additional bromine are fed (i) periodically or continuously, and (ii) individually or in admixture into the second reaction zone separately and apart from the feed of reaction product mixture formed in A); and
the amount of additional Lewis acid bromination catalyst fed into the second reaction zone is an amount that substantially continuously maintains a catalytic amount of Lewis acid bromination catalyst in the second reaction zone and the amount of bromine fed into the second reaction zone is an amount that substantially continuously maintains a stoichiometric excess of bromine in the second reaction zone.

8. A process as in claim 7 wherein said stoichiometric excess is an excess of bromine in the range of about 50 to about 150 mole percent of the amount required to perbrominate the partially brominated diphenyl oxide being fed into said second reaction zone.

9. A process as in claim 1 wherein said Lewis acid bromination catalyst utilized in the second reaction zone is aluminum chloride or aluminum bromide, or both.

10. A process as in claims 1 wherein said reaction zone in A) is a continuously stirred tank reactor.

11. A process as in claim 1 wherein in B) an inversely related time-temperature feed of reaction product mixture formed in A) into a second reaction zone is maintained substantially continuously.
